(19) United States
(12) Patent Application Publication
(10) Pub. No.: US 2023/0346253 A1
(43) Pub. Date: Nov. 2, 2023

(54) MULTI-MODAL SYSTEM AND METHOD FOR TRACKING RESPIRATORY HEALTH

(71) Applicants: GEORGIA TECH RESEARCH CORPORATION, Atlanta, GA (US); EMORY UNIVERSITY, Atlanta, GA (US)

(72) Inventors: Omer INAN, Atlanta, GA (US); Gari CLIFFORD, Atlanta, GA (US); Venu G. GANTI, Atlanta, GA (US); Samer MABROUK, Atlanta, GA (US); Mohsen SAFAEI, Atlanta, GA (US); Daniel Corbett WHITTINGSLOW, Atlanta, GA (US); David W. WRIGHT, Atlanta, GA (US)

(21) Appl. No.: 18/025,750

(22) PCT Filed: Sep. 10, 2021
(86) PCT No.: PCT/US2021/049793

§ 371 Date: Mar. 10, 2023

Related U.S. Application Data
(60) Provisional application No. 63/076,502, filed on Sep. 10, 2020.

(51) Int. Cl. A61B 5/08; A61B 5/01; G16H 40/67; G16H 50/20; A61B 5/00; A61B 7/00; G16H 50/30

(52) U.S. Cl. CPC A61B 5/0809; A61B 5/01; A61B 5/4842; A61B 5/7267; A61B 5/746; A61B 7/003; G16H 40/67; G16H 50/20; G16H 50/30; A61B 2562/0204; A61B 2562/0219

(57) ABSTRACT

A method to evaluate respiratory health includes obtaining (i) one or more lung acoustic signals and (ii) one or more bioimpedance spectroscopy signals, wherein the one or more acoustic signals and the one or more bioimpedance spectroscopy signals are concurrently acquired over multiple respiratory cycles; generating values for (i) a first set of plurality of statistical features and/or a first set of time-frequency domain features using the obtained one or more lung acoustic signals and (ii) a second set of plurality of statistical features and/or a second set of time-frequency domain features using the obtained one or more bioimpedance spectroscopy signals; and generating, using one or more trained classifiers, a respiratory health value representative of a respiratory health of the patient by application of the values of the first and second sets of plurality of statistical features and time-frequency domain features to the one or more trained classifiers.

### CROSS-REFERENCE TO RELATED PATENT APPLICATIONS

This application claims the benefit of and priority to U.S. Provisional Pat. Application No. 63/076,502, filed on Sep. 10, 2020, which is hereby incorporated by reference in its entirety.

### BACKGROUND

A major challenge in the initial assessment of patients presenting with respiratory ailments, whether in the emergency room (ER) or by emergency medical technicians (EMTs), is the timely recognition of respiratory complications, e.g., that require mechanical ventilation. For example, the novel coronavirus (i.e., COVID-19) pandemic has overwhelmed many medical centers and healthcare practitioners across the United States and could do so again in the near future as isolation mandates are relaxed and people return to normal daily activities. While current methods for assessing patients often involve clinical triaging and pulse oximeter readings to identify patients at risk for developing respiratory complications, one of the unique aspects of COVID-19 has been the acute and irreversible respiratory collapse that is often clinically unrecognized until an episode of respiratory distress. Often urgent resuscitation is further complicated and delayed due to workflows requiring the appropriate wear of personal protective equipment (PPE). Therefore, earlier identification of deterioration, through novel means of sensor technology is a critical component of preventing catastrophic events for hospitalized patients.

### SUMMARY

One implantation of the present disclosure is a method to evaluate respiratory health. The method can include obtaining, by a processor, (i) one or more lung acoustic signals using one or more contact microphones placed on a patient and (ii) one or more bioimpedance spectroscopy signals using one or more bioimpedance spectroscopy electrodes placed on the patient, where the one or more acoustic signals and the one or more bioimpedance spectroscopy signals are concurrently acquired over multiple respiratory cycles, generating, by the processor, values for (i) a first set of plurality of statistical features and/or a first set of time-frequency domain features using the obtained one or more lung acoustic signals and (ii) a second set of plurality of statistical features and/or a second set of time-frequency domain features using the obtained one or more bioimpedance spectroscopy signals, and generating, by the processor, using one or more trained classifiers, a respiratory health value representative of a respiratory health of the patient by application of the values of the first and second sets of plurality of statistical features and time-frequency domain features to the one or more trained classifiers.

In some embodiments, the one or more bioimpedance spectroscopy signals are used in a bioimpedance spectroscopy-based assessment of a lung of the patient for multi-location digital auscultation.

In some embodiments, the bioimpedance spectroscopy-based assessment includes statistical or time-frequency domain analysis.

In some embodiments, the generated respiratory health value is used to assess for a decline or an improvement in respiratory health.

In some embodiments, the method further includes causing, by the processor, a generation of an audible or visual alert when the generated respiratory health value is assessed to be declining by a pre-defined metric.

In some embodiments, the method further includes causing, by the processor, a generation of a message notification to a physician monitoring system when the generated respiratory health value is assessed to be declining by a pre-defined metric.

In some embodiments, the method further includes generating, by the processor, values for (i) a third set of plurality of statistical features and/or a first set of time-frequency domain features using an obtained one or more inertia sensing signals and (ii) a fourth set of plurality of statistical features and/or a fourth set of time-frequency domain features using an obtained one or more temperature signals, where (i) the third set of plurality of statistical features and/or the set of time-frequency domain features associated with the one or more inertia signals and/or (ii) the fourth set of plurality of statistical features and/or the set of time-frequency domain features associated with the one or more temperature signals is used with the first and second sets of plurality of statistical features and time-frequency domain features to generate the respiratory health value.

In some embodiments, the first set of plurality of statistical features and/or a first set of time-frequency domain features is derived based on an analysis selected from a group consisting of sample entropy, multiscale entropy (MSE), transfer entropy, continuous wavelet transform, fast Fourier, acoustic cepstral.

In some embodiments, the trained classifier is based on multivariate logistic regression, support vector machines, or random forests.

In some embodiments, the method further includes generating, by the processor, values for (i) a third set of plurality of statistical features and/or a third set of time-frequency domain features using an obtained one or more temperature signals, where the one or more temperature signals are concurrently acquired over the multiple respiratory cycles using one or more temperature sensors, where the third set of plurality of statistical features and/or a third set of time-frequency domain features are used by the one or more trained classifiers to determine the respiratory health value.

In some embodiments, the generated respiratory health value is used to monitor patient deteriorating respiratory health state associated with COVID-19.

In some embodiments, the generated respiratory health value is used to monitor a deteriorating respiratory health state of the patient due to pneumonia.

In some embodiments, the generated respiratory health value is used to monitor a deteriorating respiratory health state of the patient by identifying a presence of fluid in a lung of the patient.

Another implantation of the present disclosure is a system to evaluate respiratory health. The system can include a multi-modality measurement system including one or more contact microphones, one or more bioimpedance spectroscopy electrodes, and one or more accelerometers, the multi-modality measurement system being configured to concurrently acquired, over multiple respiratory cycles, (i) one or more lung acoustic signals using the one or more contact microphones, (ii) one or more bioimpedance spectroscopy signals using the one or more bioimpedance spectroscopy electrodes, and (iii) the one or more inertia sensing signals using the one or more accelerometers, and an analysis system including a processor and memory having instructions stored thereon, where execution of the instructions by the processor, causes the processor to generate values for (i) a first set of plurality of statistical features and a first set of time-frequency domain features using the obtained one or more lung acoustic signals and (ii) a second set of plurality of statistical features and a second set of time-frequency domain features using the obtained one or more bioimpedance spectroscopy signals, and generate using one or more trained classifiers, a respiratory health value associated with a respiratory health or diagnostics of the patient by application of the first, second, and third sets of plurality of statistical features and time-frequency domain features to the one or more trained classifiers.

In some embodiments, the multi-modality measurement system further includes one or more temperature sensors and/or one or one or more accelerometers, the multi-modality measurement system being configured to concurrently acquired over the multiple respiratory cycles (i) one or more temperature signals using the one or more temperature sensors or (ii) one or more inertia signals using the one or more accelerometers, where a set of plurality of statistical features and/or a set of time-frequency domain features can be computed from the one or more temperature signals or the one or more accelerometers, and where the set of plurality of statistical features and/or the set of time-frequency domain features associated with the one or more temperature signals or one or more accelerometers is used with the first and second sets of plurality of statistical features and time-frequency domain features to generate the respiratory health value.

In some embodiments, the analysis system is operatively coupled to the multi-modality measurement system over a network.

In some embodiments, the system further includes a speaker, the speaker being coupled to the measurement system to generate an audible alert from a signal generated by the processor based on the generated respiratory health value.

In some embodiments, the system further includes a display, the display being coupled to the measurement system to display visuals associated with the generated respiratory health value.

Yet another implantation of the present disclosure is a system to evaluate respiratory health. The system can include an analysis system including a processor and memory having instructions stored thereon, where execution of the instructions by the processor causes the processor to obtain, from a storage area network or a multi-modality measurement system, over a network, (i) one or more lung acoustic signals using one or more contact microphones placed on a patient, (ii) one or more bioimpedance spectroscopy signals using one or more bioimpedance spectroscopy electrodes placed on the patient, and (iii) one or more inertia sensing signals associated with the patient from one or more accelerometers placed on the patient, where the one or more acoustic signals, the one or more bioimpedance spectroscopy signals, and the one or more accelerometers have been concurrently acquired from a patient over multiple respiratory cycles, generate values for a first set of plurality of statistical features and a first set of time-frequency domain features using the obtained one or more lung acoustic signals, a second set of plurality of statistical features and a second set of time-frequency domain features using the obtained one or more bioimpedance spectroscopy signals, and a third set of plurality of statistical features and a third set of time-frequency domain features using the obtained one or more one or more inertia sensing signals, and generate, using one or more trained classifiers, a respiratory health value associated with a respiratory health or diagnostics of the patient by application of the values of the first, second, and third sets of plurality of statistical features and time-frequency domain features to the one or more trained classifiers.

In some embodiments, the analysis system further includes a network interface, the network interface being configured to receive a data file from a multi-modality measurement system, the data file having the one or more lung acoustic signals, the one or more bioimpedance spectroscopy signals, and the one or more inertia sensing signals.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a diagram of a process for tracking respiratory health using multi-modal physiological sensing, according to some embodiments.

FIG. 2 is a diagram of sensor placement on a patient for multi-modal physiological sensing, according to some embodiments.

FIG. 3 is a diagram of a wearable system for respiratory health tracking, according to some embodiments.

FIG. 4 illustrates example sensors for multi-modal physiological sensing, according to some embodiments.

FIG. 5 is a block diagram of a controller for a respiratory health tracking system, according to some embodiments.

FIGS. 6A and 6B are example layouts for the controller of FIG. 5, according to some embodiments.

FIG. 7 is a process for determining a patient’s respiratory health, according to some embodiments.

FIG. 8 is another process for determining a patient’s respiratory health, according to some embodiments.

FIG. 9 illustrates additional examples of sensor placement on a patient, according to some embodiments.

### DETAILED DESCRIPTION

Referring generally to the FIGURES, a system and methods for tracking a patient’s respiratory health are shown, according to some embodiments. In particular, the disclosed system and methods may utilize multi-modal physiological sensing techniques to determine the patient’s respiratory health. These multi-modal physiological sensing techniques may provide an inexpensive and broadly accessible option for assessing and monitoring patients with aliments that affect the respiratory system, such as COVID-19. The system described herein may capture multiple modalities of patient health characteristics relevant to the pathophysiologic effects of COVID-19; however, it will also be appreciated that the system and methods described herein are not limited to the evaluation and treatment of COVID-19 but may also be implemented to evaluate and treat other respiratory diseases.

As briefly mentioned above, the U.S. healthcare system has struggled to handle the unprecedented surge in patients requiring evaluation and monitoring for the novel coronavirus (COVID-19). Frontline healthcare workers are often tasked with triaging and screening patients under potentially dangerous conditions, such as due to inadequate supplies and training to manage this task. Current Centers for Disease Control (CDC) guidelines advise mild cases to self-isolate with supportive care and monitoring at home while more severe cases are admitted to the hospital and closely monitored. In a hospital setting, patients are kept under respiratory isolation protocol, which requires the donning and doffing of personal protective equipment (PPE) by healthcare workers every time they enter the room. Global shortages of PPE have led to suboptimal infectious control measures being instituted in hospitals throughout the nation. This highly infectious virus presents a novel threat that the current healthcare systems are poorly equipped to handle.

To address these shortcomings in the triage and monitoring of patients as well as supply shortages, new technologies must be developed that could efficiently monitor key physiologic parameters. Once available, the monitoring system can be used to monitor and identify other pathologies or conditions that can affect respiratory health. The system described herein can be made affordable and portable such that they could be used in a variety of settings, such as in an ambulance and/or emergency room in some embodiments for triage on each new patient, in a respiratory isolation room to mitigate the need for PPE and unnecessary exposure during routine exams. And even for mild cases, the exemplary system can be used to monitor patients under isolation at home, e.g., to monitor for signs of deterioration via telemedicine. Moreover, the exemplary system can additionally provide discrete interval measurements, e.g., of oxygenation and breathing rate, that can be presented to patients or clinicians to guide how care is escalated. Such more nuanced tool for continuous monitoring has the potential to help clinicians evaluate which “well appearing” patients may actually need to be prioritized for treatments (i.e., treatments that will certainly be in scarcity and inevitably be limited by the supply chain).

Indeed, multiple modality physiological sensing (e.g., from the chest), as implemented by the system and methods described herein, may provide a means of accurately screening and triaging patients. For example, COVID-19 has a clinical course that presents with mild symptoms (e.g., dry cough, dyspnea) and in approximately 15% of cases progresses to severe pneumonia with other life-threatening complications (e.g., acute respiratory distress syndrome (ARDS), aseptic shock, and/or organ dysfunction). A cardiopulmonary physical exam and vital signs are key components of the diagnosis and monitoring of this disease. Fever and cough are common clinical features, but patients may also have tachycardia, tachypnea, and, if fluid develops in the lungs (e.g., pneumonia or pleural effusion), fremitus, dullness to percussion, and egophony. In some current methods of treatment, these findings must all be determined via direct patient contact. The high transmissibility, shortage of PPE, high caseloads, and recommendations to stay at home with mild cases present challenges that a traditional medical workup is ill-suited to handle.

In some embodiments, lung sounds, fluid, and volume measurements may be obtained by the system comprising wearable contact microphones, bioimpedance spectroscopy (BIS), and kinematic measures, and may be fused by an analysis system with skin temperature for an integrated picture of COVID-19 risk and severity or other respiratory health risk. Advantageously, the system described herein may be configured as a wearable device that incorporates contact microphones, BIS, accelerometers, and temperature sensors that are placed on a patient and to provide continuous feedback on their cardiopulmonary status. The sensor system may offer several benefits over other standards of care. For example, the exemplary system can be used to synchronously capture traditional auscultatory findings of both the heart and lungs from multiple lung fields using contact microphones capable of recording wide bandwidth audio signals. The system can include accelerometers that can detect irregularities in movement indicating increased work of breathing or activity levels, and BIS that could quantify changes in pulmonary fluid levels.

Fusing multiple modalities of sensing into a single recording/monitoring system can improve specificity and sensitivity, particularly when the modalities reflect different underlying physiological / structural health parameters. COVID-19, as with many viral illnesses, has a variable presentation in patients; thus, anchoring the screening or management of these patients to one or two vital signs may be ineffective. Employing multiple complementary modalities of sensing, as provide via the system described herein, could provide a more sensitive and specific indicator of patient condition and risk of deterioration than single sensing modalities alone could capture. Specifically, as mentioned above, an acoustic array of sensors can provide multiple measurements of lung and heart sounds, thus capturing tachycardia, tachypnea, and meaningful lung sounds such as rales, crackling, and wheezing as well as the frequency and severity of cough1. A BIS approach can be additionally employed to quantify changes in fluid levels in the lung (i.e., pneumonia) and pleural space (i.e., pleural effusions) and can output impedance pneumography measures such as lung volume and respiratory flows. Temperature measurements at one or more sites can further be employed to provide for monitoring skin temperature reflective of fever, a prevalent symptom for COVID-19. In addition, inertial measurements can provide three main indicators, including enabling the assessment of irregular movements associated with increased breathing work, capturing the movements of the body to provide a means of discarding or correcting for motion artifact corrupted data and, providing a means of placing changes in the sensed parameters in the context of movement and/or postural shifts. Moreover, the relationship between each physiological parameter captured synchronously provides important information for the prediction of patient deterioration.

In some embodiments, the exemplary system is configured to quantify (e.g., in real-time) the decline of cardiopulmonary status via bioimpedance spectroscopy-based assessment of fluid in the lungs and multilocation digital auscultation. In some embodiments, this system includes a machine learning (ML) based analysis system that can provide COVID-19 risk index (e.g., designed from digital biomarkers measured by the system) and that may be used by healthcare workers (e.g., doctors, nurses, emergency medical technicians (EMTs), etc.) to rapidly and accurately assessing a patient’s status at presentation. With continued use, the longitudinal deterioration in hospitalized patients can be monitored to, for example, facilitate more effective triage and management of patients upon their arrival to a healthcare facility. Additionally, patient health can be monitored after discharge (e.g., following a positive diagnosis or mild case) to determine if conditions are deteriorating or improving, thereby informing patient management decisions.

### Multi-Modal Respiratory Health Tracking

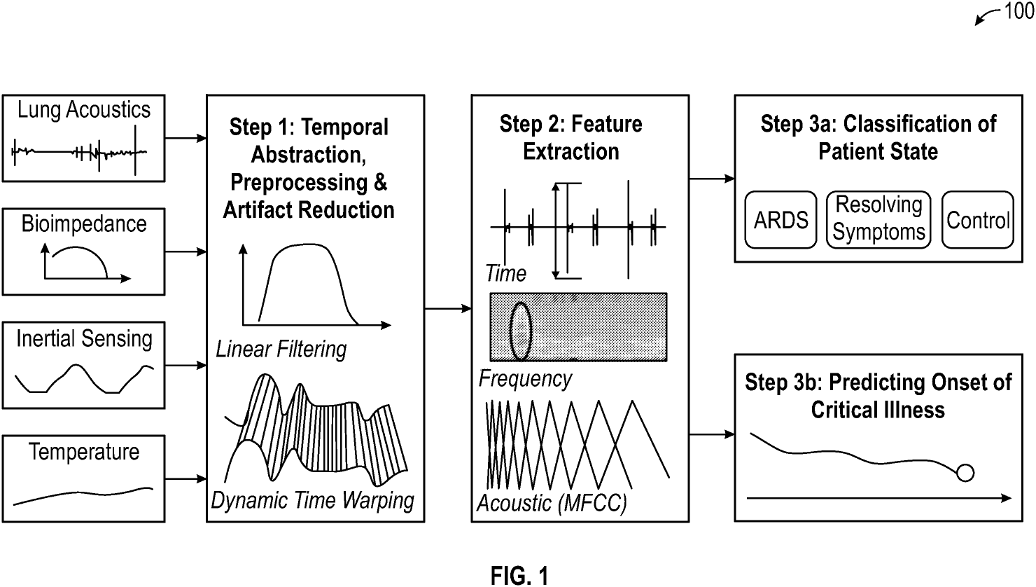

Turning first to FIG. 1, a diagram of a process 100 for tracking respiratory health using multi-modal physiological sensing is shown, according to some embodiments. Multi-modal physiological sensing, as described herein, generally includes both structural and physiological health sensing. Accordingly, process 100 may provide a number of benefits over other systems and methods of tracking respiratory health by collecting and evaluating data across a variety of different sensors measuring a variety of different physical and/or physiological parameters associated with a patient. In particular, process 100 can represent an analytical “pipeline” for extracting and/or analyzing data from multiple different sensors positioned on or near a patient.

As shown, process 100 may start with the collection of sensor data, also referred to herein as “raw” data (i.e., data that has not been manipulated). Sensor data can include but is not limited to lung acoustics, bioimpedance data, inertia data, and temperature (e.g., body temperature) data. Many other types of data may also be collected and evaluated, as discussed in greater detail below. In some embodiments, sensor data includes time series data (i.e., data collected at various points over time) or recordings sampled from measured signals provided by one or more sensors.

After collection, the raw sensor data may be preprocessed or “cleaned” for subsequent feature extraction. The pre-processing may be performed in part by the collection hardware or within the analytical pipeline. In some embodiments, preprocessing the sensor data can include filtering the data to remove out-of-band noise (i.e., noise outside of a predefined frequency band), such as via a linear filtering technique. The filtered and/or unfiltered sensor data may then be evaluated for quality using dynamic time warping. Dynamic time warping can include, for example, comparing the sensor data to existing templates of high-quality data. In some embodiments, preprocessing also includes removing outlier data points using, for example, a Mahalanobis distance technique.

At “Step 2” of process 100, features may be extracted (i.e., computed) from the preprocessed data. Features are generally data points, or measurable properties, representative of the data being processed. In process 100, for example, features that are extracted from the preprocessed lung acoustic, bioimpedance, inertia, and temperature data can include time domain, frequency domain, and acoustic (e.g., Mel Frequency Cepstral Coefficient (MFCC)) features. In some embodiments, features are extracted from windows of the time series sensor data. In some such embodiments, a window can encapsulate 200 millisecond (ms) of data, but it will be appreciated that a window for feature extraction can be any size (e.g., less than or greater than 200 ms).

After extraction, the features may be used as inputs to a trained classifier or multiple trained classifiers for determining a state of the patient (e.g., acute respiratory distress, resolving symptoms, healthy, etc.) and/or a patient’s respiratory health. A classifier is, in general, a machine learning algorithm (e.g., XGBoost) that determines a “class” or label for a given input. For example, in process 100, the features extracted from the preprocessed sensor data may be used as inputs to one or more machine learning models, where the one or more trained machine learning models are configured to output a value associated of a class (i.e., a state) that represents the patient’s respiratory health. It will be appreciated that any suitable machine learning algorithm or model may be utilized to classify outputs said features. For example, the classifier(s) may be neural networks and/or may use multivariate logistic regression, support vector machines (SVMs), random forests, etc. In some embodiments, an output of the classifier(s) is associated with a tiered indicator (e.g., red, yellow, green) which may be presented (e.g., via a user interface) to the patient or caregiver for adjusting care accordingly, as discussed in greater detail below. It should be appreciated that a similar process can be performed to provide data for the identification of features to be used to trained or develop the machine learning algorithm.

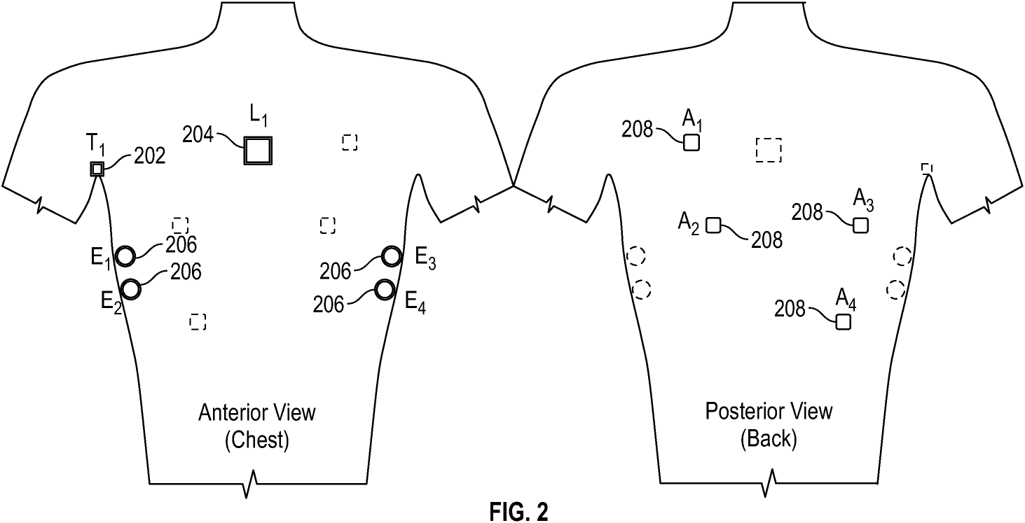

Referring now to FIG. 2, a diagram of sensor placement on a patient for multi-modal physiological sensing is shown, according to some embodiments. In particular, the diagram of FIG. 2 illustrates exemplary placement of a plurality of bioimpedance, inertia (i.e., movement), temperature, and acoustic sensors, although it will be appreciated by those in the art that other sensors may also be utilized and/or place on or near a patient. It will also be appreciated that the positions of the various sensors shown in FIG. 2 are not exact, i.e., the positions of each sensor may vary slightly from that shown in FIG. 2.

As shown, temperature sensor 202, also labeled as T₁, may be placed at or near an armpit of the patient to provide an indication of skin temperature. Temperature data may indicate, for example, whether the patient’s temperature is elevated (e.g., due to the onset of a fever). In some embodiments, a second temperature sensor (e.g., similar to or the same as temperature sensor 202, not shown) is positioned near the patient but is not in contact with the skin of the patient. This second temperature sensor may be configured to measure ambient temperature, such as the temperature of a room that the patient is occupying. Ambient temperature may, for example, confound body temperature and may also affect BIS measurements so the reading of the second temperature sensor can be used to normalize or adjust for the BIS measurements.

Also shown is an inertial measurement unit (IMU) 204, labeled as L₁, that may be placed on the patient’s sternum to detect the movements of the chest wall associated with respiratory effort. IMU 204 may also provide an indication of body position and posture by detecting gravitational acceleration. Similar to the second temperature sensor described above, a second IMU (not shown) may be utilized in combination with IMU 204 to determine body position and posture. For example, IMU 204 may detect movements of the chest wall while the second IMU detects body position, which is used as a reference to inform other measurements (e.g., bioimpedance and acoustic).

To assess changes in fluid levels in the lungs, at the alveoli or the pleural space, four electrodes 206, labeled as E₁-E₄, may also be placed on the sides of the thorax at approximately the level of the xiphoid process. In some embodiments, electrodes 206 are silver/silver chloride (Ag/AgCl) gel electrodes placed distally and proximally to the lung(s). Bioimpedance can be measured by applying a small alternating current (AC) through one or more of the electrodes 206 and measuring the voltage drop caused by this current through the remaining one or more of electrodes 206. Static full spectroscopy bioimpedance measurements of the lung may be taken at various times (e.g., hourly), and while in between measurements, dynamic multifrequency bioimpedance at low and high frequency (e.g., 5 kHz and 100 kHz) may be collected at a high sampling rate (e.g., greater than 30 Hz). Both the dynamic and static measurements may be used to assess the longitudinal changes in tidal volume and static fluid content of the lungs. In some embodiments, bioimpedance is measured across the chest at a single frequency. In other embodiments, bioimpedance sensing may be performed at multiple frequencies or through a frequency sweep (e.g., spectroscopy).

Additionally, four acoustic sensors 208, labeled as A₁-A₄, may be placed on the posterior side of the patient. Acoustic sensors 208 may be configured to acquire lung and heart sounds signals from four sites on the chest synchronously. In some embodiments, acoustic sensors 208 are configured as miniature, low-noise, wide-bandwidth accelerometers placed directly on the surface of the skin with sufficient backing force to capture sounds produced by the patient’s body accurately and repeatably.

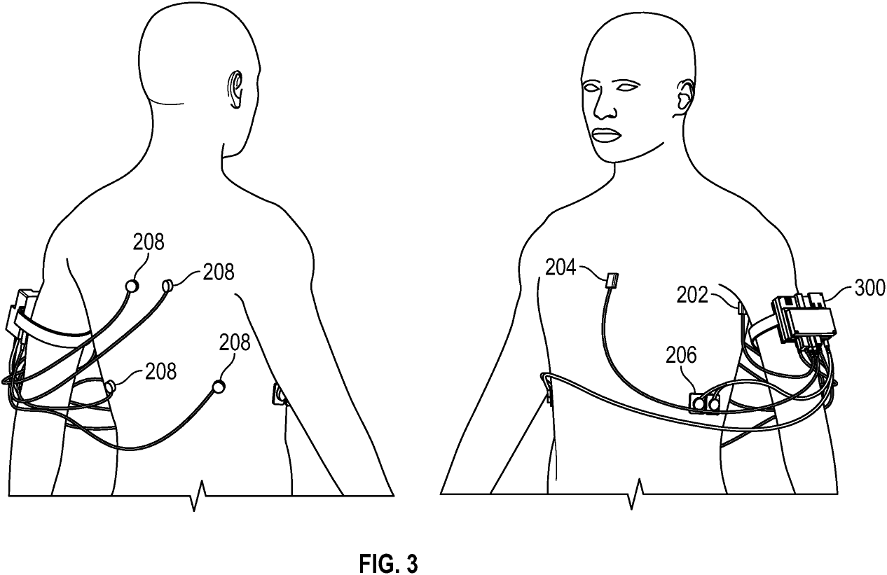

Referring now to FIG. 3, a diagram of a wearable system for respiratory health tracking is shown, according to some embodiments. Additionally, FIG. 3 illustrates sensor placement (e.g., any of sensors 202-208, described above) on a model of a patient’s torso. As shown, for example, temperature sensor 202 is positioned on the patient’s skin, at or near the patient’s armpit, and IMU 204 is positioned on the patient’s sternum. Electrodes 206 are positioned on the sides of the thorax at approximately the level of the xiphoid process. As shown in a posterior view, acoustic sensors 208 may be positioned at various points on the patient’s back. In some embodiments, acoustic sensors 208 are positioned over the middle and inferior lung lobes on the right side of the body, and over the heart and inferior lobe on the left side of the body. In some embodiments, sensors 202-208 are secured to the patient’s skin using any suitable attachment mechanism, such as a medical grade adhesive. For example, Rycote Lavalier Stickies (P/N RY065567) and/or medical grade backing tape may be used to secure sensors 202-208 to the patient.

Each of sensors 202-208 may be electrically and/or communicatively coupled to a control unit 300, such as by a series of insulated and flexible leads extending from a housing of the sensor(s) to control unit 300. Control unit 300 may be removably attached to the patient by an armband formed of, for example, an elastic material, a hook-and-loop fastener, or the like. Advantageously, these materials allow control unit 300 and sensors 202-208 to be quickly and easily attached to and/or removed from the patient. The armband may be configured such that it does not impede blood pressure cuff measurements, as it can be placed on a contralateral arm. Additionally, placement of control unit 300 on the body of the patient reduces the risk of sensors 202-208 being pulled out of place during patient movement, especially amidst emergency transit. In some embodiments, control unit 300 may be utilized without the armband shown in FIG. 3 if desired for patient comfort and/or to mitigate the creation of pressure/contact injuries in the event of prolonged use.

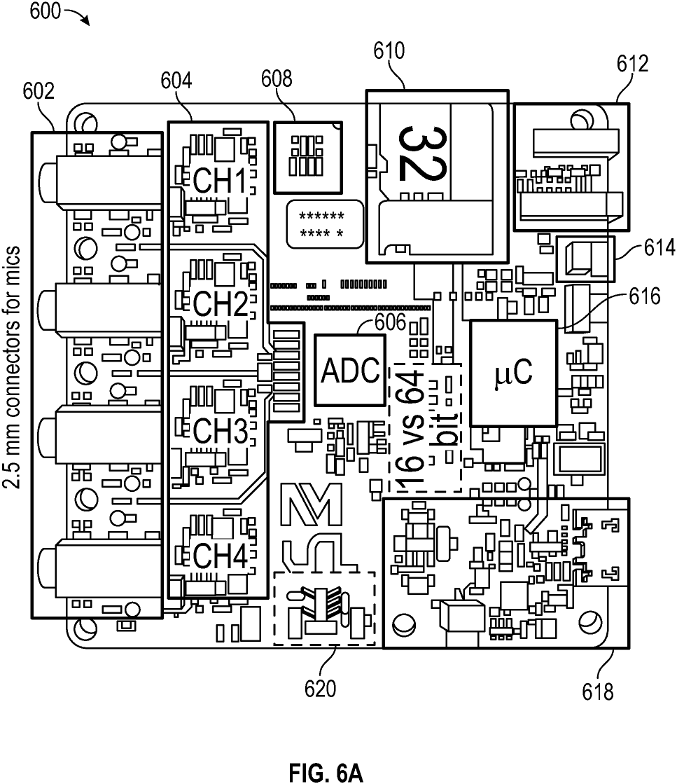

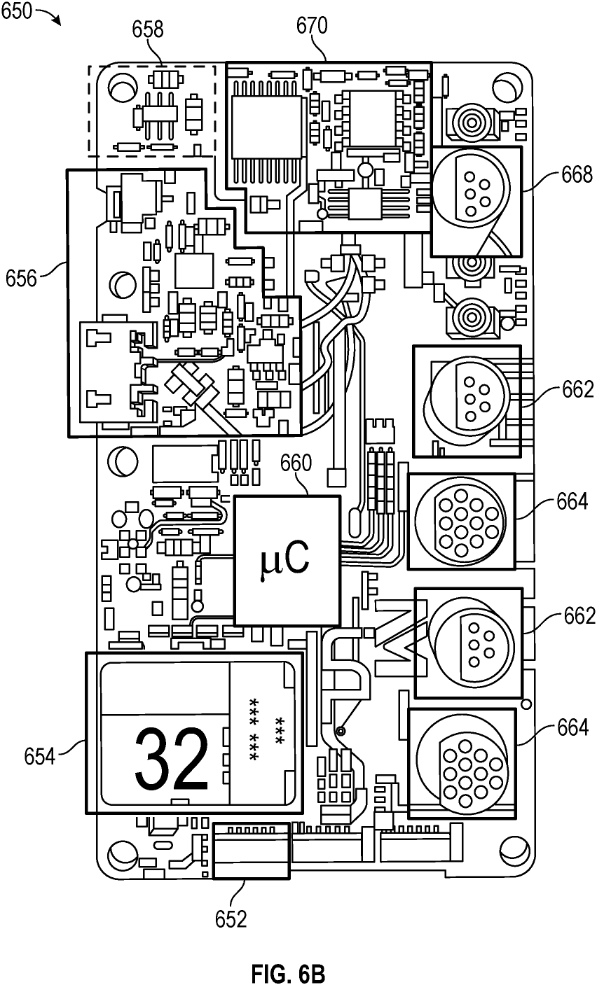

In some embodiments, control unit 300 includes a housing, which may be moisture sealed. In some such embodiments, control unit 300 may be formed of a material that is suitable for sterilization, such as via ultra-violet (UV) light, steam, alcohol, etc., so that control unit 300 may be quickly and easily sanitized for use on multiple patients. In some embodiments, the housing material and other components of control unit 300 may be constructed from lightweight materials (e.g., plastics, aluminum, titanium, etc.) to minimize the weight of control unit 300 for patient comfort. The housing of control unit 300 may contain one or more circuit boards for receiving, processing, and/or otherwise manipulating data from sensors 202-208, as discussed herein. For example, control unit 300 may include at least two circuit boards, such as the circuit board discussed below with respect to FIGS. 6A and 6B.

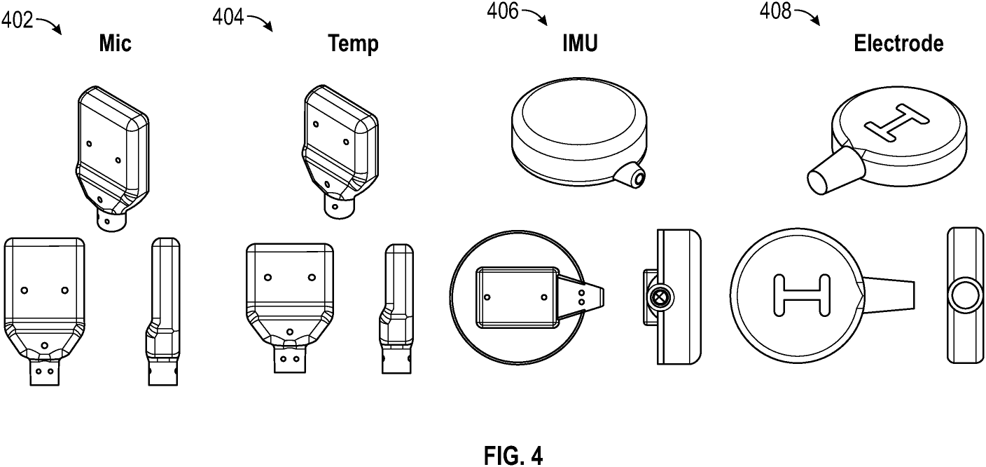

Referring now to FIG. 4, example sensors for multi-modal physiological sensing are shown, according to some embodiments. In particular, FIG. 4 may show example sensor packaging with plastic overmolding, such as for any of the sensors described above with respect to FIGS. 2 and 3 (e.g., sensors 202-208); however, it will be appreciated that the sensor packaging show in FIG. 4 is not intended to be limited and, accordingly, the sensors described herein can be of any form (e.g., size, shape, color, etc.). As shown, an acoustic sensor 402 (e.g., similar to or the same as acoustic sensors 208) may be packaged in a substantially rectangular housing measuring approximately 27.59 mm in length, 15.24 mm in width, and 6.35 mm in depth. One example of a commercially available sensor for use as acoustic sensor 402 is BU-23173-000, Knowles Electronics LLC, USA.

Likewise, a temperature sensor 404 (e.g., similar to or the same as temperature sensor 202) may be packaged in a substantially rectangular housing measuring approximately 22.77 × 15.24 × 5.84 mm. One example of a commercially available sensor for use as temperature sensor 404 is TMP116, Texas Instruments Inc., Dallas, TX. An inertial measurement unit (IMU) 406 (e.g., similar to or the same as IMU 204) may be packaged in a circular or disk-shaped housing measuring approximately 9.24 mm in radius and 7.72 mm in depth. One example of a commercially available sensor for use as IMU 406 is BMX055 Bosch Sensortec GmbH Kusterdingen, Germany.

An electrode 408 (e.g., for bioimpedance detection) may also be packaged in a circular or disk-shaped housing measuring approximately 9.54 mm in radius and 5.08 mm in depth. As described above, electrode 408 may be an Ag/AgCl gel electrode. In some embodiments, each of sensors 402-408 may be packed in a different color or type of material to differentiate the various functionalities of sensors 402-408. For example, acoustic sensor 402 may be formed of a yellow plastic overmold while temperature sensor 404 may be formed of a red plastic overmold. In some embodiments, any of sensors 402-408 may be formed of a sanitizable material. Additionally, in some embodiments, any of sensors 402-408 may be configured for single use (i.e., the sensor(s) may be disposed of after use).

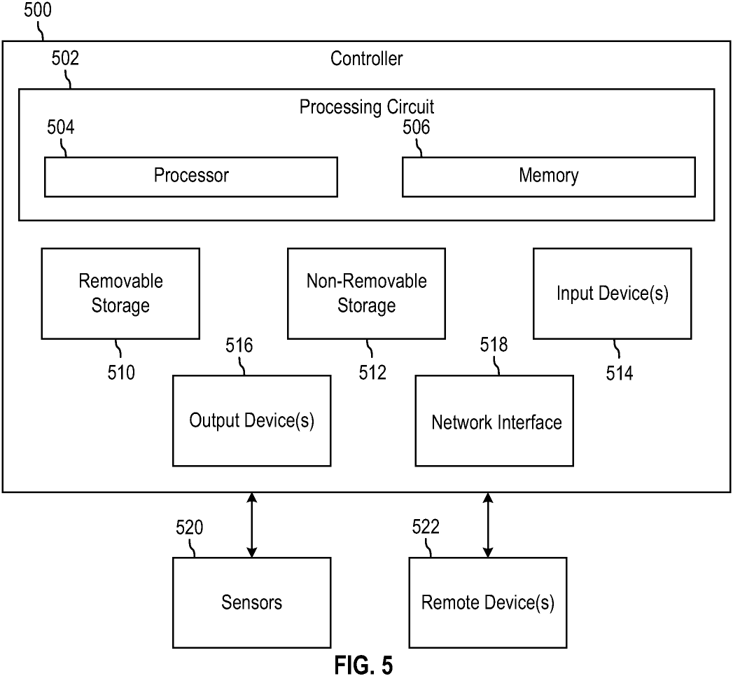

Referring now to FIG. 5, a block diagram of a controller 500 for multi-modal respiratory health tracking is shown, according to some embodiments. In some embodiments, controller 500 may be included in control unit 300 described above. Controller 500 may be configured to implement process 100 and any of the other methods described herein. At a high-level, for example, controller 500 may be configured to receive data from a plurality of sensors positioned on or near a patient, process the sensor data to determine a patient’s respiratory health, and initiate one or more automatic responses based on the determination of the patient’s respiratory health. As mentioned above, controller 500 may advantageously implement multi-modal techniques by aggregating data from multiple different types of sensors (e.g., acoustic, bioimpedance, temperature, inertia, etc.) to form determinations of respiratory health.

Controller 500 is shown to include a processing circuit 502 that includes a processor 504 and a memory 506. Processor 504 can be a general-purpose processor, an application specific integrated circuit (ASIC), one or more field programmable gate arrays (FPGAs), a group of processing components, or other suitable electronic processing components. In some embodiments, processor 504 is configured to execute program code stored on memory 504 to cause controller 500 to perform one or more operations. Accordingly, processor 504 can be communicatively coupled to memory 506 as via processing circuit 502. For example, instructions (e.g., data) may be transmitted (e.g., via processing circuit 502) from memory 506 to processor 504, at which point processor 504 may execute said instructions.

Memory 506 can include one or more devices (e.g., memory units, memory devices, storage devices, etc.) for storing data and/or computer code for completing and/or facilitating the various processes described in the present disclosure. In some embodiments, memory 506 includes tangible, computer-readable media that stores code or instructions executable by processor 504. Tangible, computer-readable media refers to any media that is capable of providing data that causes the controller 500 (i.e., a machine) to operate in a particular fashion. Example tangible, computer-readable media may include, but is not limited to, volatile media, non-volatile media, removable media and non-removable media implemented in any method or technology for storage of information such as computer readable instructions, data structures, program modules or other data.

Accordingly, memory 506 can include random access memory (RAM), read-only memory (ROM), hard drive storage, temporary storage, non-volatile memory, flash memory, optical memory, or any other suitable memory for storing software objects and/or computer instructions. Memory 506 can include database components, object code components, script components, or any other type of information structure for supporting the various activities and information structures described in the present disclosure. Memory 506 can be communicably connected to processor 504 via processing circuit 502 and can include computer code for executing (e.g., by processor 504) one or more processes described herein. In some embodiments, controller 500 may include removable storage 510 and/or non-removable storage 512, which may be similar to memory 506 for storing instructions or other data that is accessible and/or executable by processor 504. For example, removable storage 240 and non-removable storage 250 may include magnetic or optical disks or tapes, USB memory sticks, flash cards (e.g., SD cards), etc.

While shown as individual components, it will be appreciated that processor 504 and/or memory 506 can be implemented using a variety of different types and quantities of processors and memory. For example, processor 504 may represent a single processing device or multiple processing devices. Similarly, memory 506 may represent a single memory device or multiple memory devices. Additionally, in some embodiments, controller 500 may be implemented within a single computing device (e.g., one server, one housing, etc.). In other embodiments controller 500 may be distributed across multiple servers or computers (e.g., that can exist in distributed locations). For example, controller 500 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by controller 500 to provide the functionality of a number of servers that is not directly bound to the number of computers in controller 500. For example, virtualization software may provide twenty virtual servers on four physical computers.

Still referring to FIG. 5, controller 500 is shown to include input device(s) 514 and output device(s) 516. Input device(s) 514 and output device(s) 516 may include any components that allow a user (e.g., a healthcare worker, a patient, etc.) to interact with controller 500. In some embodiments, input device(s) 514 include keyboards, keypads, switches, dials, mice, track balls, touch screens, voice recognizers, card readers, paper tape readers, or other types of components that allow the user to input data and/to make selections for controller 500. Output device(s) 516 can include printers, video monitors, liquid crystal displays (LCDs), LED displays, touch screen displays, other displays, speakers, etc., for displaying data to the user. In one example, controller 500 may include a touchscreen user interface that both displays data to the user (e.g., as graphics) and receives user inputs (e.g., via a touch). Thus, in this example, a touchscreen user interface may be considered both an input and an output device.

Controller 500 is also shown to include a network interface 518 for communicating (i.e., transmitting and receiving) data with external devices. Network interface 518 may include a modem, modem bank, Ethernet card, universal serial bus (USB) interface card, serial interface, token ring card, fiber distributed data interface (FDDI) card, wireless local area network (WLAN) card, radio transceiver card such as code division multiple access (CDMA), global system for mobile communications (GSM), long-term evolution (LTE), worldwide interoperability for microwave access (WiMAX), and/or other air interface protocol radio transceiver card, or any other type of network device.

In some embodiments, controller 500 is configured to communicate (i.e., transmit and receive data) with sensors 520, which may include any of the sensors described above (e.g., sensors 202-208 and/or sensors 402-408). In some such embodiments, sensors 520 may be wired and/or wirelessly coupled to controller 500 to facilitate data exchange. For example, sensors 520 may be communicably coupled to controller 500 via a series of flexible cables, as described above. Accordingly, controller 500 may receive sensor data and can store the received data on any of memory 506, removable storage 510, and non-removable storage 512. Received and/or stored sensor data may subsequently be manipulated by processor 504 according to any of the processes described herein.

In some embodiments, controller 500 is also configured to communicate (i.e., transmit and receive data) with remote device(s) 522, such as via network interface 518. Remote device(s) 522 may include any devices (e.g., computing devices) that are external or remote to controller 522. In some embodiments, remote device(s) 522 can include remote displays or user interfaces, speakers, etc. In some embodiments, remote device(s) 522 can include external servers configured to perform any of the processes described herein. For example, in some such embodiments, controller 500 may be configured to collect data from sensors 520 and subsequently transmitted to a remote computing device (e.g., a server) to reduce the required computing power provided by controller 500 and/or to reduce energy usage by controller 500.

In some such embodiments, controller 500 can be considered a multi-modality measurement system for collecting sensor data and, in such embodiments, may be configured to transmit sensor data to a remote analysis system for processing. For example, controller 500 may be configured to transmit, via network interface 518, a data file containing acoustic sensor data, BIS sensor data, IMU data, and/or temperature data. Accordingly, in some embodiments, controller 500 may be configured to receive data (e.g., after processing), such as control decisions, from the remote analysis system. In some embodiments, remote device(s) 522 may include computing devices associated with a healthcare worker (e.g., a doctor, a nurse) and/or the patient. For example, controller 500 may transmit data to and/or receive data from a smartphone, tablet, computer, etc., belonging to a healthcare worker or patient.

Referring now to FIGS. 6A and 6B, example layouts for controller of 500 are shown, according to some embodiments. In particular, FIGS. 6A and 6B are diagrams showing the layout of various components of controller 500 on a circuit board or other similar device; however, it will be appreciated that the layout of controller 500 is not limited to the examples shown in these figures. In some embodiments, the circuit boards of FIGS. 6A and 6B may be utilized cooperatively in controller 500. For example, a first circuit board 600 shown in FIG. 6A may be configured to collect and process audio data from acoustic sensors (e.g., acoustic sensors 208 or microphones) while a second circuit board 650 may be configured to collect and process bioimpedance and/or BIS data, inertial data, and temperature readings. It will also be appreciated that, in some embodiments, all of the features described below with respect to circuit boards 600 and 650 may be included on a single circuit board or may be distributed across additional boards.

Turning first to FIG. 6A, circuit board 600 is shown to include a plurality of connectors 602 for communicably coupling one or more acoustic sensors (e.g., acoustic sensors 208 or microphones). For example, each of connectors 602 may be 2.5 mm connectors for receiving analog audio data from the one or more acoustic sensors. Analog audio data may then be preprocessed by one or more audio processing circuits 604. As shown, for example, each of audio processing circuits 604 may correspond with a particular audio channel, associated with at least one acoustic sensor. In some embodiments, 16-bit audio data is captured at a 46 kHz sample rate. After preprocessing, the analog audio signals may be converted to digital signals (i.e., data) by an analog-to-digital converter (ADC) 606.

Circuit board 600 is also shown to include light emitting diodes (LEDs) 608. LEDs 608 may include any number and/or color of LEDs for providing visual indications to a user. For example, LEDs may indicate a status of circuit board 600 and/or a power source (e.g., on/off, charging, processing data, receiving/transmitting data, etc.). Circuit board 600 can also include removable storage 610, shown here as a microSD card, although any suitable type of removable storage may be included. One or more switches 612 may be configured to turn circuit board 600 on/off (e.g., by connecting or removing power) and may control other functions described herein. For example, one of switches 612 may “lock” circuit board 600 by preventing the reading and/or writing of data (e.g., programming) to an internal memory device.

Still referring to FIG. 6A, circuit board 600 also includes a sync connector 614 for coupling circuit boards 600 and 650. In particular, sync connector 614 may transmit data and/or clock signals between circuit boards 600 and 650 to ensure that circuit boards 600 and 650 operate cooperatively. At the heart of circuit board 600 is a processing circuit 616 (e.g., a microcontroller), which may be the same as or functionally similar to processing circuit 502 described above. In particular, processing circuit 616 may be configured to receive and process the digital audio signals from ADC 606. Also shown are power and data connectors 618 and 620, which can include a battery, a battery charging circuit, digital and analog power circuits, and a microUSB or other type of data connector.

Turning now to FIG. 6B, circuit board 650 is also shown to include a sync connector 652 for coupling circuit boards 600 and 650. Circuit board 650 also includes removable storage 654, shown here as a microSD card, although any suitable type of removable storage may be included. Also shown are power and data connectors 656 and 658, which can include a battery, a battery charging circuit, digital and analog power circuits, and a microUSB or other type of data connector. Unlike circuit board 600, however, circuit board 650 also includes a plurality of temperature sensor connectors 662 for communicably coupling to one or more temperature sensors (e.g., temperature sensor 202). As described above, for example, a first temperature sensor may be positioned under an armpit of the patient while a second temperature sensor may be included in a housing for controller 500 to provide a reference temperature.

Circuit board 650 also includes a plurality of IMU connectors 664 for communicably coupling to one or more IMUs (e.g., IMU 204). For example, a first IMU may be positioned on a sternum of the patient while a second IMU may be included in a housing for controller 500 to provide a reference measurement. Also shown is a BioZ connector 668 for communicably coupling one or more bioimpedance sensors (e.g., electrodes 206). In some embodiments, circuit board 650 includes a BioZ analog front-end 670, which includes components for receiving and processing signals from the one or more bioimpedance sensors. For example, BioZ analog front-end 670 may be configured to convert analog signals (e.g., measured voltage values) into digital signals for processing by a processing circuit 660. Additionally, processing circuit 660 may receive data from any number of temperature sensors and IMUs, which may be processed and/or manipulated as described herein.

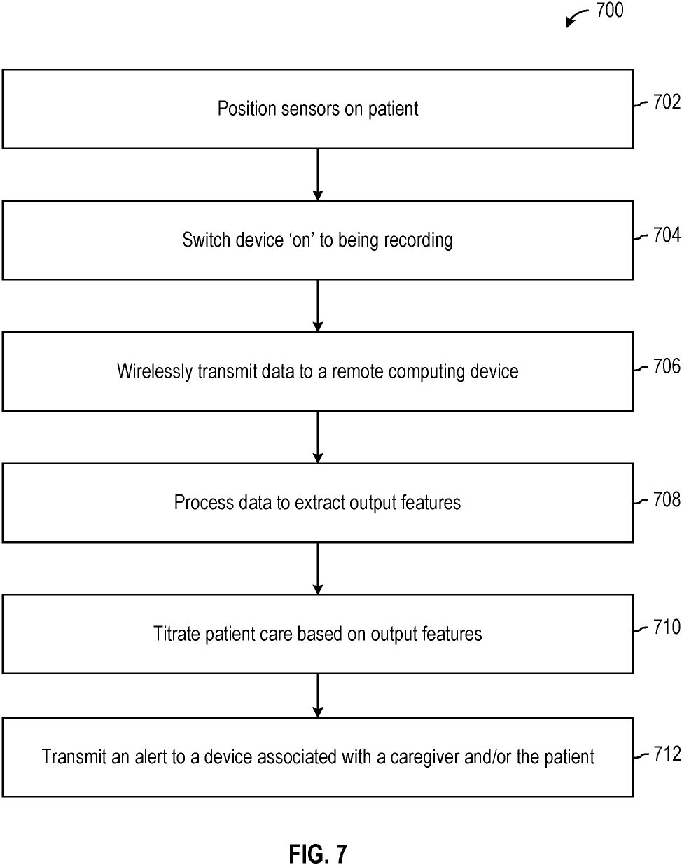

Referring now to FIG. 7, a process 700 for determining a patient’s respiratory health in a healthcare setting is shown, according to some embodiments. In some embodiments, process 700 is implemented by controller 500, as described above. At a high level, process 700 includes positioning one or more sensors on a patient (e.g., sensors 202-208), recording one or more corresponding physiological parameters for the patient, and processing the recorded data to titrate patient care. Advantageously, process 700 may utilize measurements from a plurality of different sensors to determine a patient’s respiratory health. It will be appreciated that certain steps of process 700 may be optional and, in some embodiments, process 700 may be implemented using less than all of the steps.

At step 702, one or more sensors are positioned on a patient. As described above, these one or more sensors may include at least acoustic sensors (e.g., microphones), bioimpedance or BIS sensors (e.g., electrodes), temperature sensors, and IMUs. The one or more sensors may, in particular, be removably attached to the skin of the patient at various points on the patient’s upper body. For example, the sensors may be attached using a suitable adhesive or adhesive strip(s). Desirable positioning of the one or more sensors is described above with respect to FIGS. 2 and 3. Additionally, at this step, each of the one or more sensors may be communicably coupled to a controller (e.g., controller 500), either via one or more flexible wires or wirelessly.

At step 704, the controller is switched ‘on’ (i.e., is powered) to being recording data from the one or more sensors. In particular, the controller may receive analog and/or digital data from the one or more sensors either continuously, or at regular time intervals (e.g., every second). For example, sensors that provide analog data may be continuously monitored while certain digital sensors may provide digital data at regular intervals. Over time, this data may be collected and, in some cases, stored on the controller. In some embodiments, sensor data is collected over multiple respiratory cycles. Unlike many other methods, sensor data may be collected concurrently from the multiple different sensors over these multiple respiratory cycles. For example, data may be collected over a minute, multiple minutes, an hour, etc.

At step 706, at least a portion of the collected data may be wirelessly transmitted to a remote computing device (e.g., remote device(s) 522). The remote computing device, or multiple computing devices, may include remote servers, etc., for processing the collected sensor data. Additionally, or alternatively, sensor data may be transmitted to a computing device associated with a healthcare worker and/or the patient. For example, a doctor may be able to view raw sensor data from the controller. In some embodiments, a copy of the collected data is transmitted to a remote computing device for storage. For example, raw sensor data may be stored for additional processing, such as at any point after collection.

At step 708, the sensor data is processed (e.g., by the controller and/or the remote computing device) to extract output features. As described above, features may include measurable properties representative of the sensor data. For example, time domain, frequency domain, and acoustic features may be extracted from the sensor data. In some embodiments, the sensor data is processed to extract a set of statistical features, or features defined through statistical analysis. In some such embodiments, the set of statistical features may be extracted by moving a window across a set of time-series data. In this case, the sensor data may be considered time-series data, as it is collected at regular intervals over time. In some embodiments, a window can encapsulate 200 ms of data, but it will be appreciated that a window for feature extraction can be any size (e.g., less than or greater than 200 ms).

In some embodiments, features are extracted using time-frequency analysis techniques. Time-frequency analysis extracts features from the sensor data (i.e., signals) in both the time and frequency domains, simultaneously. For example, features may be extracted using a short-time Fourier transform (STFT) or other suitable technique. In some embodiments, either prior to or concurrent with feature extraction, BIS data (e.g., data from one or more bioimpedance sensor) is used in a bioimpedance spectroscopy-based assessment of a lung of the patient for multi-location digital auscultation. Accordingly, the bioimpedance spectroscopy-based assessment can include one or both of a statistical or time-frequency domain analysis of the BIS data.

At step 710, patient care is titrated based on the output features. In particular, the extracted features may be used as inputs to a classifier or multiple classifiers for determining a state of the patient (e.g., acute respiratory distress, resolving symptoms, healthy, etc.) and/or a patient’s respiratory health. The classifier(s) may be any suitable machine learning algorithm or model (e.g., neural network) for classifying said features. For example, the classifier(s) may be neural networks and/or may use multivariate logistic regression, support vector machines (SVMs), random forests, extreme gradient boosting (i.e., XGBoost), etc. The classifier(s) may output a tiered indicator (e.g., “red,” “yellow,” “green”) that indicates a status (i.e., respiratory health) of the patient. For example, a “green” status may indicate that the patient has adequate or relatively normal lung function, while a “red” status may indicate severely degraded respiratory health. Accordingly, a patient with a “red” or severe status may require an escalation in treatment or may be triaged over a patient with a “green” status.

At step 712, an alert may be transmitted to a remote device, such as a computing device (e.g., phone, tablet, laptop, etc.) associated with a healthcare worker and/or the patient. In some embodiments, the alert indicates the patient’s status (i.e., respiratory health), such as by providing sensor data, an indication of the patient’s classification, etc. In some embodiment, the alert may be transmitted to a healthcare worker if the patient’s condition is critical and/or worsening (e.g., “red”). Accordingly, in some embodiments, an alert is not transmitted if the patient’s status is unchanged but not normal (e.g., “yellow”), improving, or relatively normal (e.g., “green”). In some embodiments, the alert may be displayed on a displayed on the healthcare worker or patient’s device, such as via a user interface that includes a screen, lights, etc.

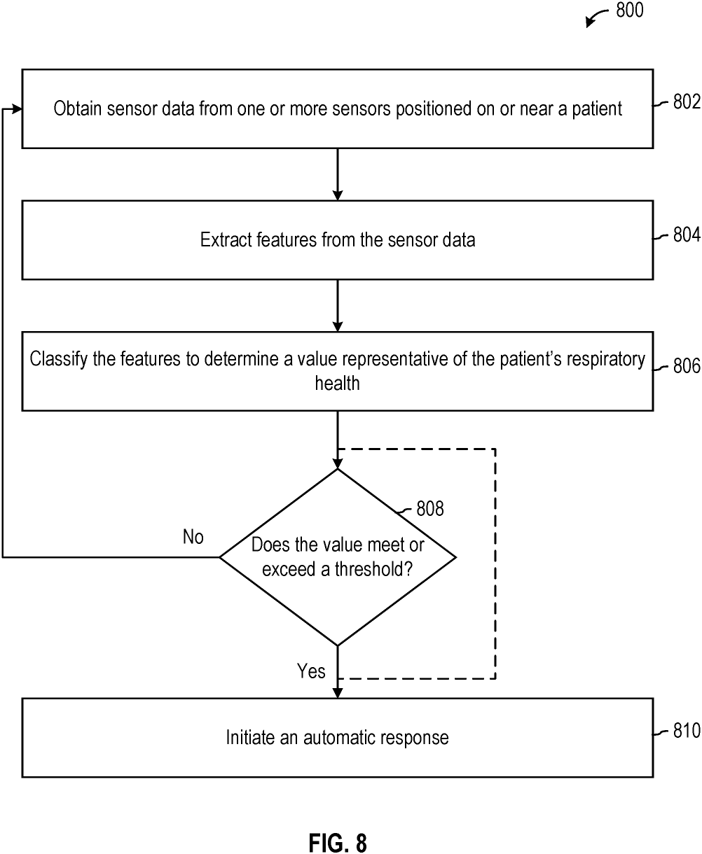

Referring now to FIG. 8, another process 800 for determining a patient’s respiratory health in a home care setting is shown, according to some embodiments. In some embodiments, process 800 is implemented by controller 500, as described above. Like process 700, process 800 may advantageously utilize measurements from a plurality of different sensors to determine a patient’s respiratory health. Specifically, process 800 involves the generation of a value indicative of the patient’s respiratory health. This value may be used to assess improvement and/or declination in the patient’s respiratory health over time, such as due to pneumonia, COVID-19, or other respiratory ailments that may cause the build-up of fluid in the lungs. Additionally, process 800 may include initiating one or more automatic response processes based on the determination of the patient’s respiratory health, to improve patient recovery and/or to triage multiple affected patients. It will be appreciated that certain steps of process 800 may be optional and, in some embodiments, process 800 may be implemented using less than all of the steps.

At step 802, sensor data is obtained from one or sensors positioned on or near a patient. Similar to step 702, described above, the one or more sensors can include at least acoustic sensors (e.g., contact microphones), bioimpedance (i.e., BIS) sensors (e.g., electrodes), temperature sensors, and IMUs, although it will be appreciated that other types of sensors may also be considered. The one or more sensors may, in particular, be removably attached to the skin of the patient at various points on the patient’s upper body, as described above with respect to FIGS. 2 and 3. Additionally, at this step, each of the one or more sensors may be communicably coupled to a controller (e.g., controller 500), either via one or more flexible wires or wirelessly.

In some embodiments, the sensor data is collected by the controller over a time period, which may be predefined. For example, the time period may be an evaluation period that is one minute, five minutes, an hour, etc., or sensor data may be continuously collected for monitoring the patient. In this example, the patient may wear the controller and/or the one or more sensors for only a short period of time (e.g., for diagnosis in a hospital setting), or the patient may wear the system continuously and/or over a longer period of time (e.g., for continued monitoring at home). In any case, sensor data may be collected over multiple respiratory cycles to ensure accuracy (e.g., accounting for outliers).

After collection, in some embodiments, the sensor data may be preprocessed or “cleaned.” In some such embodiments, preprocessing can include filtering the data to remove out-of-band noise (i.e., noise outside of a predefined frequency band), such as via a linear filtering technique. The filtered and/or unfiltered sensor data may then be evaluated for quality using dynamic time warping. Dynamic time warping can include, for example, comparing the sensor data to existing templates of high-quality data. In some embodiments, preprocessing also includes removing outlier data points using, for example, a Mahalanobis distance technique. In some embodiments, preprocessing can also include converting analog signals (e.g., from acoustic sensors) to digital data.

At step 804, the raw and/or preprocessed sensor data is further processed to extract statistical and/or time-frequency domain features. As described above at step 708, features may include measurable properties representative of the sensor data, such as time domain, frequency domain, and/or acoustic features. In some embodiments, a set of statistical features may be extracted by moving a window across a set of time-series data. In some such embodiments, the window can encapsulate any amount of data, such as 200 ms, but it will be appreciated that the window for feature extraction can be any size (e.g., less than or greater than 200 ms). In some embodiments, time-frequency domain features are using a short-time Fourier transform (STFT) or other suitable techniques. In other embodiments, one or both of the statistical and time-frequency domain features are extracted using sample entropy, multiscale entropy (MSE), transfer entropy, continuous wavelet transform, fast Fourier, or acoustic cepstral techniques, among other. In some embodiments, either prior to or concurrent with feature extraction, BIS data (e.g., data from one or more bioimpedance sensor) is used in a bioimpedance spectroscopy-based assessment of a lung of the patient for multi-location digital auscultation. Accordingly, the bioimpedance spectroscopy-based assessment can include one or both of a statistical or time-frequency domain analysis of the BIS data.

As an example, lung acoustic signals, bioimpedance spectroscopy (BIS) signals, inertia sensing signals, and/or temperature data may be obtained at step 802 and may subsequently (e.g., either before or after preprocessing) be analyzed using any of the techniques described above. In particular, a first set of statistical and/or time-frequency domain features may be extracted (i.e., generated) from the lung acoustic data, a second set of statistical and/or time-frequency domain features may be extracted from the BIS data, a third set of statistical and/or time-frequency domain features may be extracted from the inertia data, and a fourth set of statistical and/or time-frequency domain features may be extracted from the temperature data.

At step 806, the extracted features are classified to determine a value representative of the patient’s respiratory health. In particular, the extracted features may be used as inputs to one or more classifiers, which are configured to output the value; however, in some embodiments, the classifier(s) may output another value which is aggregated to determine the value representing the patient’s respiratory health. As described above, the classifiers may be trained ML models (e.g., neural networks, etc.) that determine a “class” or label based on a given input. For example, the trainer classifier(s) may include multivariate logistic regression, support vector machine (SVM), or random forest models, among others. As designated herein, a classifier may be “trained” using a known good dataset. For example, data (e.g., lung acoustics, BIS data, etc.) may be collected from a first group of patients that are known to have health lungs (i.e., no respiratory ailments) and/or from a second group of patients that are known to have a respiratory ailment, such as pneumonia, COVID-19, etc. The classifier(s) may be trained by using the known good data as an input and comparing it to known outputs (e.g., the patient’s known diagnosis). In some embodiments, the classifier(s) may be retrained or continuously trained over time, to improve predictions. For example, each classification output by the classifier may be compared to the patient’s diagnosis (e.g., at a later time) to determine the accuracy of the classification.

In some embodiments, the trained classifiers may only require a portion of the available sensor data to determine a classification. For example, in some cases, a determination of lung function may be made using only acoustic and BIS. In particular, acoustic and BIS data may be the most beneficial or indicative of lung health. For example, acoustic and BIS data may readily indicate the presence of fluid in the lungs. In some embodiments, outputs may be improved by considering other data, such as temperature and inertia data, but these data points may not necessarily be required to determine the patient’s respiratory health.

In some embodiments, the classifier(s) may output a value that indicates a status (i.e., respiratory health) of the patient. For example, the classifier may output a numerical, alphanumeric, or other value, such as a “red,” “yellow,” “green” or “severe,” “moderate,” “good” classification. In this example, a “green” status may indicate that the patient has adequate or relatively normal lung function, a “yellow” status may indicate that the patient has deteriorate lung function that is not worsening, and a “red” status may indicate severely degraded or deteriorating respiratory health. Accordingly, a patient with a “red” or severe status may require an escalation in treatment or may be triaged over a patient with a “green” status.

In some embodiments, the value indicating the patient’s respiratory health may be compared to one or more threshold values to determine whether additional care is needed (step 808), although it will be appreciated that this step is optional in process 800. As an example, the threshold may be a numeric value (e.g., % of normal lung capacity) or a specific classification (e.g., “red”) that, when met or exceeded by the value output by the classifier(s), indicates that the patient’s respiratory health is severe or deteriorating.

At step 810, one or more automatic response processes may be initiated based on the patient’s respiratory health classification. In some embodiments, the automatic response processes include generating an audible or visual alert that can be presented on the respiratory health tracking system (e.g., controller 500) or a device associated with a healthcare worker, caregiver, and/or the patient. For example, a light and/or display of controller 500 may indicate the alert, such as by flashing or presenting the alert on a user interface. As another example, the alert may be transmitted to a remote device (e.g., a smartphone) and may cause the remote device to flash a light, display a message, make a noise, vibrate, etc. In this example, controller 500 may cause an email, a text message, a voice call, etc., to be transmitted to a healthcare worker or patient’s device. In some embodiments, the alert may cause a speaker (e.g., internal to controller 500 or external) to generate an audible alert (e.g., a tone). In some embodiments, an alert is only generated responsive to the value meeting or exceeding a threshold or pre-defined metric. For example, a healthcare worker may only be alerted if a patient’s respiratory health is declining.

In some embodiments, the automatic response processes include transmitting control signals to one or more external devices (e.g., remote device(s) 522) to affect care or treatment provided to the patient. For example, a control signal may be transmitted to a ventilator to increase or decrease airflow. As another example, a control signal may be transmitted may be transmitted to an electronic hospital bed, causing the bed to actuate to incline the patient (e.g., from a lying position). In some embodiments, the automatic response processes include presenting, via a user interface, a display including visuals (e.g., colors, graphs, text, etc.) that indicate the patient’s respiratory health or diagnostics.

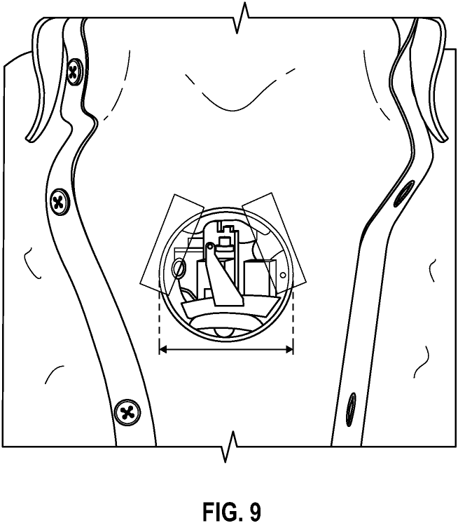

Referring now to FIG. 9, additional examples of sensor placement on a patient are shown, according to some embodiments. FIG. 9, for example, shows the placement of a wearable device on the chest of a patient. This wearable device may be an IMU, such as IMU 204 described above, or may be a device for measuring cardiogenic vibrations (e.g., in patients with heart failure). As shown, the wearable device may be secured to the skin of the patient with one or more adhesive strips.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

What is claimed is:

1. A method to evaluate respiratory health comprising:
obtaining, by a processor, (i) one or more lung acoustic signals using one or more contact microphones placed on a patient and (ii) one or more bioimpedance spectroscopy signals using one or more bioimpedance spectroscopy electrodes placed on the patient, wherein the one or more acoustic signals and the one or more bioimpedance spectroscopy signals are concurrently acquired over multiple respiratory cycles;
generating, by the processor, values for (i) at least one of a first set of a plurality of statistical features and a first set of time-frequency domain features using the obtained one or more lung acoustic signals, and (ii) at least one of a second set of a plurality of statistical features and a second set of time-frequency domain features using the obtained one or more bioimpedance spectroscopy signals; and
generating, by the processor, using one or more trained classifiers, a respiratory health value representative of a respiratory health of the patient by application of the values of the first and second sets of plurality of statistical features and time-frequency domain features to the one or more trained classifiers.

2. The method of claim 1, wherein the one or more bioimpedance spectroscopy signals are used in a bioimpedance spectroscopy-based assessment of a lung of the patient for multi-location digital auscultation.

3. The method of claim 2, wherein the bioimpedance spectroscopy-based assessment comprises statistical or time-frequency domain analysis.

4. The method of claim 1, wherein the generated respiratory health value is used to assess for a decline or an improvement in respiratory health.

5. The method of claim 4 further comprising:
causing, by the processor, a generation of an audible or visual alert when the generated respiratory health value is assessed to be declining by a pre-defined metric.

6. The method of claim 3 further comprising:
causing, by the processor, a generation of a message notification to a physician monitoring system when the generated respiratory health value is assessed to be declining by a pre-defined metric.

7. The method of claim 1, further comprising:
generating, by the processor, values for (i) a third set of plurality of statistical features and/or a first set of time-frequency domain features using an obtained one or more inertia sensing signals and (ii) a fourth set of plurality of statistical features and/or a fourth set of time-frequency domain features using an obtained one or more temperature signals,
wherein (i) the third set of plurality of statistical features and/or the set of time-frequency domain features associated with the one or more inertia signals and/or (ii) the fourth set of plurality of statistical features and/or the set of time-frequency domain features associated with the one or more temperature signals is used with the first and second sets of plurality of statistical features and time-frequency domain features to generate the respiratory health value.

8. The method of claim 1, wherein the first set of plurality of statistical features and/or a first set of time-frequency domain features is derived based on an analysis selected from a group consisting of sample entropy, multiscale entropy (MSE), transfer entropy, continuous wavelet transform, fast Fourier, acoustic cepstral.

9. The method of claim 1, wherein the trained classifier is based on multivariate logistic regression, support vector machines, or random forests.

10. The method of claim 1 further comprising:
generating, by the processor, values for (i) a third set of plurality of statistical features and/or a third set of time-frequency domain features using an obtained one or more temperature signals, wherein the one or more temperature signals are concurrently acquired over the multiple respiratory cycles using one or more temperature sensors,
wherein the third set of plurality of statistical features and/or a third set of time-frequency domain features are used by the one or more trained classifiers to determine the respiratory health value.

11. The method of claim 1, wherein the generated respiratory health value is used to monitor patient deteriorating respiratory health state associated with COVID-19.

12. The method of claim 1, wherein the generated respiratory health value is used to monitor a deteriorating respiratory health state of the patient due to pneumonia.

13. The method of claim 1, wherein the generated respiratory health value is used to monitor a deteriorating respiratory health state of the patient by identifying a presence of fluid in a lung of the patient.

14. A system to evaluate respiratory health, the system comprising:
a multi-modality measurement system comprising one or more contact microphones, one or more bioimpedance spectroscopy electrodes, and one or more accelerometers, the multi-modality measurement system being configured to concurrently acquired, over multiple respiratory cycles, (i) one or more lung acoustic signals using the one or more contact microphones, (ii) one or more bioimpedance spectroscopy signals using the one or more bioimpedance spectroscopy electrodes, and (iii) the one or more inertia sensing signals using the one or more accelerometers; and
an analysis system comprising a processor and memory having instructions stored thereon, wherein execution of the instructions by the processor, causes the processor to:
generate values for (i) a first set of plurality of statistical features and a first set of time-frequency domain features using the obtained one or more lung acoustic signals and (ii) a second set of plurality of statistical features and a second set of time-frequency domain features using the obtained one or more bioimpedance spectroscopy signals; and
generate using one or more trained classifiers, a respiratory health value associated with a respiratory health or diagnostics of the patient by application of the values of the first, second, and third sets of plurality of statistical features and time-frequency domain features to the one or more trained classifiers.

15. The system of claim 14, wherein the multi-modality measurement system further comprises one or more temperature sensors and/or one or one or more accelerometers, the multi-modality measurement system being configured to concurrently acquired over the multiple respiratory cycles (i) one or more temperature signals using the one or more temperature sensors or (ii) one or more inertia signals using the one or more accelerometers,
wherein a set of plurality of statistical features and/or a set of time-frequency domain features can be computed from the one or more temperature signals or the one or more accelerometers, and wherein the set of plurality of statistical features and/or the set of time-frequency domain features associated with the one or more temperature signals or one or more accelerometers is used with the first and second sets of plurality of statistical features and time-frequency domain features to generate the respiratory health value.

16. The system of claim 14, wherein the analysis system is operatively coupled to the multi-modality measurement system over a network.

17. The system of claim 14 further comprising:
a speaker, the speaker being coupled to the measurement system to generate an audible alert from a signal generated by the processor based on the generated value.

18. The system of claim 14 further comprising:
a display, the display being coupled to the measurement system to display visuals associated with the generated respiratory health value.

19. A system to evaluate respiratory health comprising:
an analysis system comprising a processor and memory having instructions stored thereon, wherein execution of the instructions by the processor causes the processor to:
obtain, from a storage area network or a multi-modality measurement system, over a network, (i) one or more lung acoustic signals using one or more contact microphones placed on a patient, (ii) one or more bioimpedance spectroscopy signals using one or more bioimpedance spectroscopy electrodes placed on the patient, and (iii) one or more inertia sensing signals associated with the patient from one or more accelerometers placed on the patient, wherein the one or more acoustic signals, the one or more bioimpedance spectroscopy signals, and the one or more accelerometers have been concurrently acquired from a patient over multiple respiratory cycles;
generate values for a first set of plurality of statistical features and a first set of time-frequency domain features using the obtained one or more lung acoustic signals, a second set of plurality of statistical features and a second set of time-frequency domain features using the obtained one or more bioimpedance spectroscopy signals, and a third set of plurality of statistical features and a third set of time-frequency domain features using the obtained one or more one or more inertia sensing signals; and
generate, using one or more trained classifiers, a respiratory health value associated with a respiratory health or diagnostics of the patient by application of the values of the first, second, and third sets of plurality of statistical features and time-frequency domain features to the one or more trained classifiers.

20. The system of claim 19, wherein the analysis system further comprising a network interface, the network interface being configured to receive a data file from a multi-modality measurement system, the data file having the one or more lung acoustic signals, the one or more bioimpedance spectroscopy signals, and the one or more inertia sensing signals.
